(19) United States
(12) Patent Application Publication
(10) Pub. No.: US 2022/0151677 A1
(43) Pub. Date: May 19, 2022

(54) Implant Electrode Systems and Methods of Providing Non-Invasive Radiofrequency Ablation and Stimulation

(71) Applicant: Blaine Cameron, Plantation, FL (US)

(72) Inventor: Blaine Cameron, Plantation, FL (US)

(21) Appl. No.: 17/529,209

(22) Filed: Nov. 17, 2021

Related U.S. Application Data
(63) Continuation of application No. 63/114,635, filed on Nov. 17, 2020.

(51) Int. Cl. A61B 18/12; A61N 1/05; A61B 18/14; A61N 1/06

(52) U.S. Cl. CPC A61B 18/1206; A61N 1/0502; A61B 2018/00577; A61N 1/0558; A61N 1/06; A61B 18/14

(57) ABSTRACT

An implanted electrode system that can be accessed to perform functions including but not limited to radiofrequency ablation, dorsal horn stimulation, peripheral nerve stimulation and bone stimulation. The implant electrode system generally includes is to have implantable devices that will allow for post-operative percutaneous radiofrequency ablation.

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### CROSS-REFERENCE TO RELATED APPLICATIONS

This is a continuation application of Blaine Cameron, Intrathecal Needle Closed System and Method of Reducing Post Dural Puncture Headache and Infection, U.S. Appl. No. 63/114,635, filed Nov. 17, 2020 and incorporated herein by reference in its entirety.

### STATEMENT REGARDING FEDERALLY SPONSORED RESEARCH OR DEVELOPMENT

Not Applicable

### THE NAMES OF PARTIES TO A JOINT RESEARCH AGREEMENT

Not Applicable

### INCORPORATION-BY-REFERENCE OF MATERIAL SUBMITTED ON A COMPACT DISC

Not Applicable

### BACKGROUND OF THE INVENTION

### Field of the Invention

Many orthopedic and orthopeasic procedures are performed for arthritis and involve implantation of devices. For example, as of 2010 over 600,000 TKA were being performed annually in the US. This is expected to increase 143 percent by 2050. Over 95 percent of TKA in the United States are performed for osteoarthritis. As of 2018 650,00 to 700,000 spine surgeries performed per year. Often these patients have pain post operatively. One option for treatment of this pain is radiofrequency ablation. For example, radiofrequency ablation of the lumbar facets, genicular nerve radiofrequency ablation, hip and shoulder radiofrequency ablation.

Radiofrequency ablation involves targeting the aforementioned using fluoroscopy. An electrical current is then run through the needle and exits through a pad placed on the patient. The problem is placement post operatively involves altered anatomy as well as navigating the hardware. The hardware itself can act as a conductor thus making radiofrequency ablation more challenging.

### DESCRIPTION OF THE RELATED ART

### Summary of the Invention

Example embodiments in general relate to implant electrode systems and more specifically it relates to an implant electrode system that can be accessed to perform functions including but not limited to radiofrequency ablation, dorsal horn stimulation, peripheral nerve stimulation and bone stimulation.

One object is to provide an implanted electrode system that can be accessed to perform functions including but not limited to radiofrequency ablation, dorsal horn stimulation, peripheral nerve stimulation and bone stimulation.

Another object is to provide an implant electrode system that facilitates radiofrequency ablation of e.g. medial branch of the dorsal primary rami percutaneously or other e.g. genicular nerve. Another object is to provide an implant electrode system including a pedicle screw to function in the traditional capacity that has an attached implant electrode system that facilitates post-operative radiofrequency ablation of the dorsal primary rami for pain relief e.g. treat post laminectomy syndrome.

Another object is to provide an implant electrode system that repeat radiofrequency ablation without needle violation of the skin is possible. Thus, reducing the risks of infection and bleeding and reducing the need to stop anticoagulation therapy thus reducing the risk of CVA and MI.

Another object is to provide an implant electrode system that reduces incidence of bleeding in patients on anticoagulation for radiofrequency ablation.

Another object is to provide an implant electrode system that reduces the incidence of infection in radiofrequency ablation in e.g. post laminectomy patients, failed total knee and failed total hip implants.

Another object is to provide an implant electrode system that reduce the incidence in stroke and myocardial infarction by eliminating the need for anticoagulation cessation in post-operative laminectomy patients.

Another object is to provide an implant electrode system that improve patient experience by pain reduction in post laminectomy radiofrequency ablation of spinal facets, failed total knee ablation of genicular nerve and failed total hip radiofrequency ablation.

Another object is to provide an implant electrode system that has a peripheral nerve stimulator function. TENS unit function and its attendant benefits.

Another object is to provide an implant electrode system that allows Spinal cord stimulator function or dorsal root ganglion stimulator function and its benefits.

Another object is to provide an implant electrode system that has an intradiscal radiofrequency function.

Another object is to provide an implant electrode system that has a bone stimulator function.

Other objects and advantages of the various embodiments of the present invention will become obvious to the reader and it is intended that these objects and advantages are within the scope of the present invention. To the accomplishment of the above and related objects, this invention may be embodied in the form illustrated in the accompanying drawings, attention being called to the fact, however, that the drawings are illustrative only, and that changes may be made in the specific construction illustrated and described within the scope of this application.

Although the invention is illustrated and described herein as embodied as an Implant Electrode System, the invention is not limited to the details shown because various modifications and structural changes may be made without departing from the invention and the equivalents of the claims. However, the construction and method of operation of the invention together with additional objects and advantages thereof will be best understood from the following description of specific embodiments when read in connection with the accompanying drawings.

### BRIEF DESCRIPTION OF THE DRAWINGS

Features and advantages of the present invention will become apparent to those skilled in the art from the following description with reference to the drawings, in which:

Example embodiments will become more fully understood from the detailed description given herein below and the accompanying drawings, wherein like elements are represented by like reference characters, which are given by way of illustration only and thus are not limitative of the example embodiments herein.

FIG. 1: is a front view of the present invention. Radiofrequency targets.

Pelvis, Greater trochanter, acetabulum, neck of femur, implant, lesser trochanter, femoral nerve, obturator nerve.

FIG. 2: is a side view of the present invention. Spinal nerve, facet joint, posterior dorsal ramus, pedicle screw.

FIG. 3: is a front view of the present invention. Genicular nerve, superior medial, superior lateral, plastic spacer, inferior medial, medial retinacular.

Lateral view Inferior patellar, inferior medial genicular nerve, superior lateral and medial genicular nerve, medial retinacular.

FIG. 4: is a rear view of the present invention. Shoulder implant.

### DETAILED DESCRIPTION OF THE INVENTION

### A. Overview

Turning now descriptively to the drawings, in which similar reference characters denote similar elements throughout the several views, the figures illustrate an example embodiment comprising is to have implantable devices that will allow for post-operative percutaneous radiofrequency ablation.

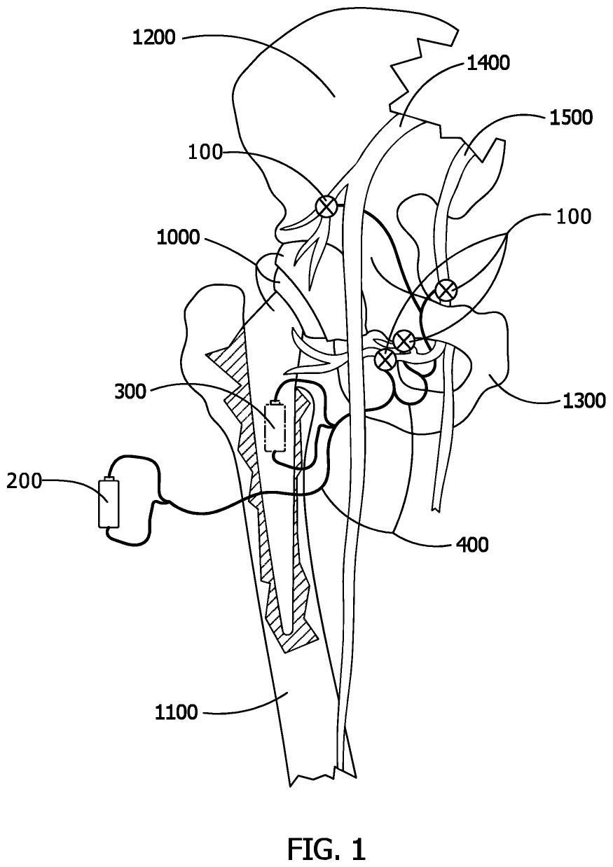

FIG. 1 shows: radio frequency target 100; external power control 200; internal power control 300; wires 400; femur implant 1000; femur 1100; ilium 1200; pubis 1300; femoral nerve 1400; and, obturator nerve 1500. Femur implant 1000 is implanted within femur 1100. Internal power control 300 and external power control 200 are connected by wires 400 to at least one or more radio frequency target 100. As illustrated the at least one or more radio frequency target 100 may be located at the femoral nerve 1400; obturator nerve 1500 or other targets within proximity to the femur implant 1000.

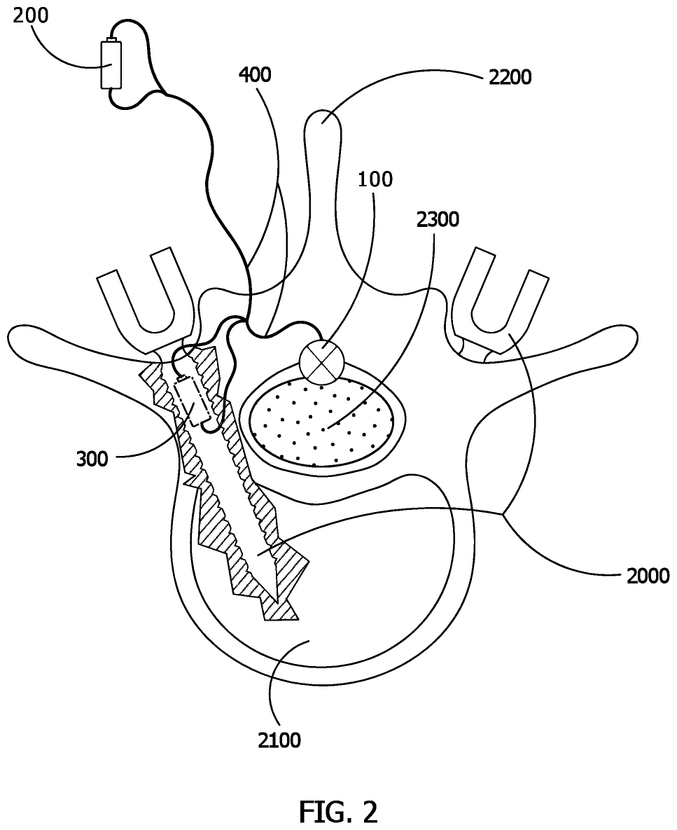

FIG. 2 shows: radio frequency target 100; external power control 200; internal power control 300; wires 400; pedicle screw 2000; vertebral body 2100; spinous process 2200; and, spinal cord 2300. External power control 200 and internal power control 300 are connected to wires 400 that are associated with at least one or more radio frequency target 100. The radio frequency target 100 may be located at the spinous process 2200, spinal cord 2300, and or adjacent to where they intersect. In the illustrated embodiment the internal power control 300 is housed within the pedicle screw 2000. The internal power control 300 may include a power source and the components for communication with an external remote and/or device. The internal power control 300 may also be preprogrammed to control the timing, amplitude and frequency of ablation.

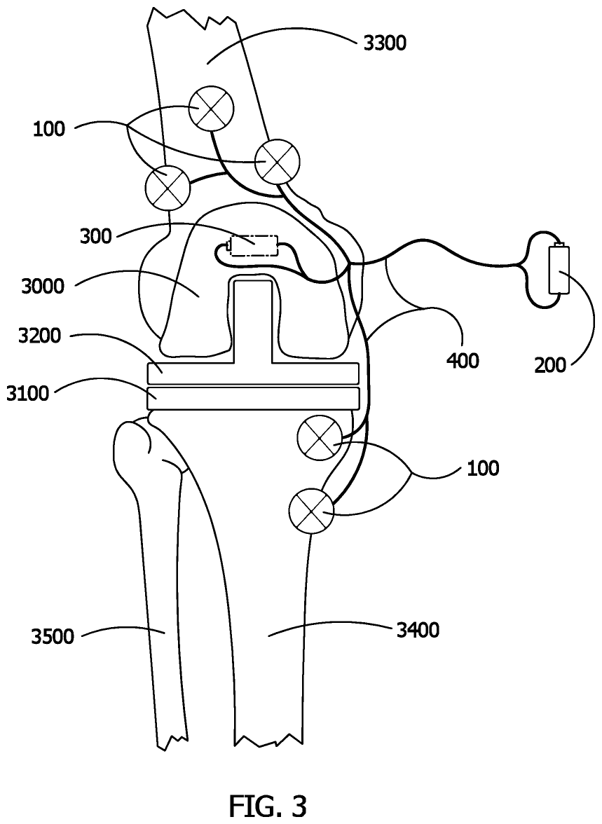

FIG. 3 shows: radio frequency target 100; external power control 200; internal power control 300; wires 400; femoral component of knee implant 3000; tibial component of knee implant 3100; plastic spacer of knee implant 3200; femur 3300, tibia 3400; and fibula 3500. Internal power control 300 and external power control 200 are connected by wires 400 to one or more radio frequency target 100. As illustrated the one or more radio frequency target 100 may be located in or adjacent to the femur 3300, tibia 3400; and fibula 3500 and surrounding nerves and tissue. The internal power control 300 may be housed within and/or adjacent to the femoral component of knee implant 3000.

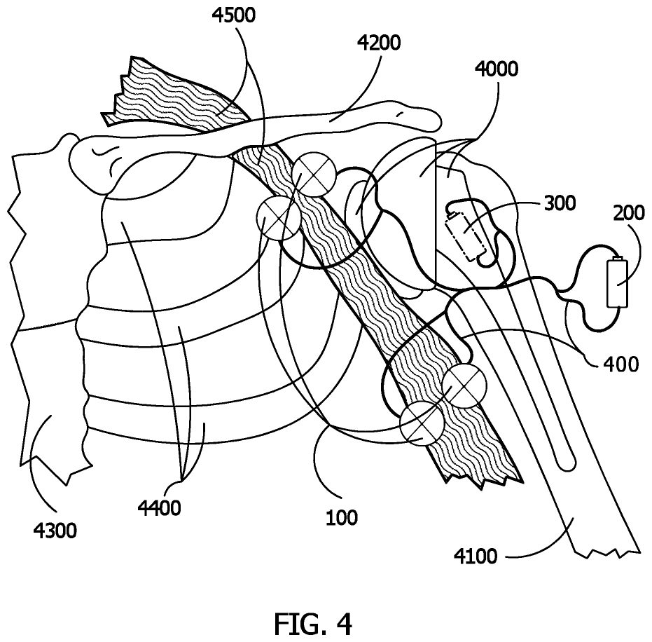

FIG. 4 shows: radio frequency target 100; external power control 200; internal power control 300; wires 400; shoulder implant 4000; humerus 4100; clavicle 4200; sternum, 4300; ribs 4400; and, brachial nerves 4500. Shoulder implant 4000 is implanted in proximity of the intersection of humerus 4100 and clavicle 4200. Internal power control 300 and external power control 200 are connected by wires 400 to one or more radio frequency target 100. As illustrated the one or more radio frequency target 100 are preferably located at the brachial nerves 4500 and also in proximity to the ribs 4400.

### B. Radiofrequency Ablation Compatible Pedicle Screw 2000

The pedicle screw 2000 comprises a contact positioned in proximity of the dorsal primary rami. The pedicle screw 2000 is generally shielded to not cause an aberrant conduction and thus focus heat and thus ablation on the medial branch of the dorsal primary rami.

The contact can be bipolar. The contact may be activated using an external wires 400 less device. The contact may be charged wirelessly.

The pedicle screw 2000 has the attendant attributes and functionality of current pedicle screws with the addition of a radiofrequency contact that is positioned to facilitate future external radiofrequency ablation of the medial branch of the dorsal primary rami. The contact can be activated externally and can be charged externally. It will be able to perform a sensory and motor current to ensure safety and give feedback to external device. Radiofrequency ablation will then be accomplished by generating heat to ablate the targeted nerve as is currently the standard of care. The difference being the contact communicates and is activated wirelessly making the future procedure noninvasive and specifically not requiring the placement of needles. This makes any future procedures noninvasive and repeatable which minimizes trauma to Os, musculature and reduces risk of infection or bleeding. This solves the need to stop or hold anticoagulation, reducing CVA or MI risk from current.

The contact may be bipolar. The implant may be shielded to prevent aberrant conduction or current dispersion. The device may be charged wirelessly to facilitate repeat procedures and not require invasive battery changing.

### C. Radiofrequency Ablation Total Knee

The implant is generally that of the usual total knee implant but contains attachments with contacts. The contacts are placed on the three branches of the genicular nerve. Superior medial, superior lateral, inferior medial, inferomedial and medial retinacular.

The total knee implant is like the knee implants and not necessary to preserve or remove PCL. However, the implant includes one or more contacts placed on branches of the genicular nerve. May include medial retinacular and inferior patellar. These contacts facilitate non-invasive radiofrequency ablation of the genicular nerve. The contact may be charged wirelessly. The contact can be activated in sensory, motor or lesion mode wirelessly. This makes any future procedures noninvasive and repeatable which minimizes trauma to Os, musculature and reduces risk of infection or bleeding. This eliminates the need to stop or hold anticoagulation, reducing CVA or MI risk from current.

The contact may be bipolar, the contact may have a sensory, motor and or lesion mode. The contact may be charged wirelessly or receive current transdermally or wirelessly.

### D. Radiofrequency Ablation Total Hip

The device consists of a hip implant with a contact placed. The targets are the articular branches of the obturator nerve and femoral nerve articular branch. These can be bipolar and accessed externally and charged wirelessly.

The contacts are placed intraoperatively at the radiofrequency targets intraoperatively at the femoral and obturator nerve as per standard of care for radiofrequency ablation. This makes any future procedures noninvasive and repeatable which minimizes trauma to Os, musculature and reduces risk of infection or bleeding. This eliminates the need to stop or hold anticoagulation, reducing CVA or MI risk from current.

The contact may be bipolar. The implant may be shielded to prevent aberrant conduction or current dispersion. The device may be charged wirelessly to facilitate repeat procedures and not require invasive battery changing.

### E. Radiofrequency Ablation Compatible Shoulder Implant

This embodiment consists of a shoulder implant with leads that are placed on the radiofrequency target nerves. These leads can be bipolar and can be accessed wirelessly externally. Additionally, these can be charged wirelessly.

This makes any future procedures noninvasive and repeatable which minimizes trauma to Os, musculature and reduces risk of infection or bleeding. This eliminates the need to stop or hold anticoagulation, reducing CVA or MI risk from current.

The contact may be bipolar. The implant may be shielded to prevent aberrant conduction or current dispersion. The device may be charged wirelessly to facilitate repeat procedures and not require invasive battery changing.

### F. Peripheral Nerve Stimulation

The implant electrode system provides peripheral nerve stimulation.

The implant electrode system is able to perform peripheral nerve stimulation. For example, when it is implanted near genitofemoral nerve.

The implant may consist of just the tip contact and thus be implanted via needle injection onto or near the desired peripheral nerve.

### G. Spinal Cord Stimulation Dorsal Root Ganglion Stimulation

The implant electrode system is able to stimulate the dorsal root ganglion.

The implant electrode can be placed during the spinal surgery in such a way so that post operatively it can be externally accessed to perform dorsal root ganglion stimulation.

The implant electrode system can be placed in such a way as to stimulate lumbricals and other desired targeted musculature.

### H. Bone Stimulation

The implant electrode system is able to provide bone stimulation to improve bone growth and healing post implantation.

The implant electrode system is placed during hip and knee or lumbar surgery to perform bone stimulation post operatively to facilitate bone growth and thus healing and implant fusion, etc.

The implant electrode system may be placed on the cortex or alternatively may be intra medullary depending on efficacy.

### I. Connections of Main Elements and Sub-Elements of Invention

The device has a remote. The remote is able to communicate with the device. Data such as temperature, timer etc. are delivered to the remote. Charging can be wirelessly accomplished. Additionally, the device can be activated and used in various mode using the remote.

### J. Alternative Embodiments of Invention

The device can be unipolar or bipolar. The device may or may not have a non-corrodible battery. The battery may be contained for example within the pedicle screw 2000 head or alternatively the charge may be sent wirelessly directly to the implant electrode system for the external remote device. The device may have one mode, lesion or may have up to three or more modes. Including but not limited to sensory mode, motor mode, and lesion mode. The frequency and amplitude may also be adjusted remotely.

The device may be able to access one contact at a time or alternatively multiple contacts simultaneously allowing for example multilevel radiofrequency ablation of facets simultaneously.

Another alternate embodiment is to have the contacts contain a predetermined amount of fluid or liquid which can be released when desired to perform a cooled radiofrequency or coolief ablation.

Another alternate of the contact is to have a peripheral stimulator or spinal cord stimulator functionality, dorsal horn stimulation or bone stimulation. The implant electrode system can be either multimodal able to do radiofrequency ablation, spinal cord stimulation, peripheral nerve stimulation or bone stimulation. Or the implant electrode system can be tailored to the functionality desired. For example, a knee implant with only a bone stimulator implant electrode device.

### K. Operation of Preferred Embodiment

An advantage of the radiofrequency implants are that they factor for the aforementioned challenges. The bipolar leads are placed surgically and are an attachment to the implant.

These bipolar implants allow for not only precise placement. They also allow for ablation remotely without the need of a needle. This makes the procedure less invasive and therefore reduces the risks of the procedure such as bleeding and infection. The bipolar implants can be accessed and charged externally.

The implant electrode system can be either multimodal able to do radiofrequency ablation, spinal cord stimulation, peripheral nerve stimulation or bone stimulation. Or the implant electrode system can be tailored to the functionality desired. For example, a knee implant with only a bone stimulator, implant electrode device.

Unless otherwise defined, all technical and scientific terms used herein have the same meaning as commonly understood by one of ordinary skill in the art to which this invention belongs. Although methods and materials similar to or equivalent to those described herein can be used in the practice or testing of the implant electrode system, suitable methods and materials are described above. All publications, patent applications, patents, and other references mentioned herein are incorporated by reference in their entirety to the extent allowed by applicable law and regulations. The implant electrode system may be embodied in other specific forms without departing from the spirit or essential attributes thereof, and it is therefore desired that the present embodiment be considered in all respects as illustrative and not restrictive. Any headings utilized within the description are for convenience only and have no legal or limiting effect.

Having thus described preferred embodiments, it should be apparent to those skilled in the art that certain advantages of the described system have been achieved. It should also be appreciated that various modifications, adaptions, and alternative embodiments thereof may be made within the scope and spirit of the present invention. The invention is further defined by the following claims.

What is claimed is:

1. An implant electrode system comprising:
at least one radio frequency target;
at least one of an internal power control and an external power control;
at least one wire; and,
at least one pedicle screw.

2. An implanted electrode device comprising.
at least one radio frequency target;
at least one of an internal power control and an external power control;
at least one wire; and,
at least one pedicle screw.

3. A method of providing non-invasive radiofrequency ablation, bone stimulation, or nerve stimulation comprising:
using an implanted electrode device comprising:
at least one radio frequency target;
at least one of an internal power control and an external power control;
at least one wire; and,
at least one pedicle screw
wherein the at least on wire delivers radio frequency ablation to a site located at a radio frequency target.
